(19) United States
(12) Patent Application Publication
(10) Pub. No.: US 2005/0202569 A1
(43) Pub. Date: Sep. 15, 2005

(54) Integrated microchemical device

(75) Inventors: Yoshiki Sakaino, Asaka-shi (JP); Yukio Sudo, Minami-Ashigara-shi (JP); Yoshihiko Abe, Asaka-shi (JP)

Correspondence Address: BIRCH STEWART KOLASCH & BIRCH, PO BOX 747, FALLS CHURCH, VA, 22040-0747, US

(73) Assignee: Fuji Photo Film Co., Ltd.

(21) Appl. No.: 11/075,434

(22) Filed: Mar. 9, 2005

(30) Foreign Application Priority Data
Mar. 10, 2004 (JP) P.2004-066800

(51) Int. Cl. G01N 33/00

(52) U.S. Cl. 436/169; 422/57

(57) ABSTRACT

A microchemical device capable of promoting mixing of a specimen and a reagent particularly in analyzing trace constituents of liquid such as blood or body fluid, and a microchemical device that can rapidly and easily analyze one or more constituents from a small amount of specimen, are provided, which is an integrated microchemical device for analyzing one or more constituents of a specimen, the microchemical device comprising at least the two elements of (a) one or more flow channels through which a specimen passes and (b) a multilayer dry analysis element that can come in contact with at least one of the flow channels and analyze the constituents of the specimen.

### TECHNICAL FIELD

This invention relates to a microchemical device for analyzing constituents in liquid of blood, body fluid, urine, etc. The invention also relates to an analysis method of trace constituents in a microspecimen using a microchemical device or a microreactor.

### BACKGROUND ART

A reactor having a microflow channel, namely, a microscale flow channel is collectively called “microreactor” and has been greatly developed in recent years (non-patent document 1: “Microreactor” written by W. Ehrfeld, V. Hessel, and H. Lowe, first edition, WILEY-VCH, 2000).

The microchemical device is used as a microfluid device appropriately formed with a structure of a microflow channel (mainly, equivalent diameter 1 mm or less) in member or a reaction tank, an electrophoretic column, a film separation mechanism, etc., connected to the flow channel, for example. The microchemical device has an internal capillary flow channel and can be used as a microreaction device (microreactor) of chemistry, biochemistry, etc., for example, as a microanalysis device such as an integrated-type DNA analysis device, a microelectrophoretic device, or a microchromatography device, an analysis specimen preparation microdevice of mass spectral, liquid chromatography, etc., a physical and chemical treatment device of extraction, film separation, dialysis, etc., a microarray manufacturing spotter, etc.

The microreactor of a microchemical device having a flow channel (passage) with equivalent diameter 1 mm or less is sit in the limelight as a new microanalysis technique also satisfying rapidity and simplicity from the points that the specimen amount can be lessened and that the device can be miniaturized. Particularly, application of the microreactor to analysis of trace constituents existing blood, urine, tissue, etc., is expected. A small integrated health care device using the microreactor is proposed (for example, patent document 1: JP-A-2001-258868).

However, in the microreactor, usually, behavior different from that of a macro reactor using liquid as a specimen is shown often as a defect.

For example, if an attempt is made to measure the activity of an enzyme in blood, to pour blood into the microreactor, perform enzyme reaction with the substrate of the enzyme in the microreactor, and measure the result, it is necessary to rapidly mix the specimen (blood) containing the enzyme and the substrate. However, the Reynolds number decreases in the microflow channel of the microreactor, etc., and thus liquid may form a laminar flow, inhibiting rapid progress of mixing; this is a problem. This problem occurs noticeably particularly in a plurality of reactions or reaction wherein a plurality of reagents need to be mixed.

Thus, to apply the microchemical device or the microreactor to microanalysis, the problem of inhibiting rapid progress of mixing a specimen and a reagent has become evident.

### DISCLOSURE OF THE INVENTION

It is an object of the invention to provide a microchemical device for promoting mixing of a specimen and a reagent particularly in analyzing trace constituents of liquid such as blood or body fluid in the microchemical device.

It is another object of the invention to provide a microchemical device that can rapidly and easily analyze one or more constituents from a small amount of specimen.

As a result of devoting ourselves to solving the above-described problems, the inventor et al. found out that mixing of a specimen and a reagent in a microchemical device is promoted by using a multilayer dry analysis element to detect a reaction, and embodied the invention.

That is, the objects of the invention are accomplished according to the following configurations:

1. An integrated microchemical device for analyzing one or more constituents in a specimen, said integrated microchemical device comprising:
(a) one or more flow channels through which a specimen passes; and
(b) a multilayer dry analysis element that can come in contact with at least one of the one or more flow channels and analyze the one or more constituents of the specimen.

2. The integrated microchemical device as described in the item 1, wherein the one or more flow channel has a width of 1 to 3000 μm.

3. The integrated microchemical device as described in the item 1 or 2, which further comprises a pretreatment element for pretreating a specimen.

4. An method of analyzing one or more constituents in a specimen, which comprises using the integrated microchemical device described in any one of the items 1 to 3.

In the invention, a multilayer dry analysis element is used in a detection system of a microchemical device as a reagent for detecting constituents of a specimen.

The dry analysis element is an analysis element using dry chemistry. In the invention, a multilayer dry analysis element is used in the detection system of the microchemical device, whereby one or more constituents can be analyzed rapidly and easily from a small amount of specimen. Accordingly, it is made possible to promote mixing of a specimen and a reagent in the microchemical device. Since it is not necessary to mix a reagent and the water content of a specimen, the problem involved when liquid and liquid are mixed in a microspace can be eliminated. The microchemical device is effective particularly if a plurality of reagents are incorporated in the microspace in the microchemical device, particularly if the microchemical device is used for multilayer analysis with a plurality of reagents incorporated in one or more layers. As the reagent is stable in a dry state and a reaction proceeds only with the water content of a specimen, rapid detection is made possible.

The microchemical device of the invention can rapidly and easily analyze one or more constituents from a small amount of specimen. There can be provided the microchemical device for promoting mixing of a specimen and a reagent particularly in analyzing trace constituents of liquid such as blood, urine or body fluid.

### BRIEF DESCRIPTION OF THE DRAWINGS

FIG. 1 is a schematic drawing of one embodiment of a flow channel in a microchemical device of the invention;

FIG. 2 is a schematic drawing of a mode of a flow channel in a microchemical device as a comparative example;

FIG. 3 is a sectional view to show one embodiment in the microchemical device of the invention; and

FIG. 4 is a sectional view to show a mode in the microchemical device as the comparative example.

### BRIEF DESCRIPTION OF REFERENCE NUMERALS

1 Specimen pouring port
2 Flow channel
3 Reaction port
4 Specimen pouring port
5 Reagent pouring port
6 Flow channel
7 Reaction port

### BEST MODE FOR CARRYING OUT THE INVENTION

In the specification, “-” indicates the range including the numeric values described preceding and following “-” as the minimum value and the maximum value.

A microchemical device for analyzing one or more constituents of a specimen in the invention is an integrated microchemical device having at least the following two elements
(a) One or more flow channels through which a specimen passes; and
(b) a multilayer dry analysis element that can come in contact with at least one of the flow channels and analyze the constituents of the specimen.

To begin with, (b) the multilayer dry analysis element that can come in contact with at least one of the flow channels and analyze the constituents of the specimen will be discussed.

<(b) Multilayer Dry Analysis Element that can come in Contact with at Least one of the Flow Channels and Analyze the Constituents of the Specimen>

The multilayer dry analysis element mentioned in the invention means a dry analysis element incorporating all or some reagents required for qualitative/quantitative analysis of constituents of the specimen such as non-measurement constituents of blood, into one or more layers. It is an analysis element using so-called “dry chemistry” Specifically, as an example of such an analysis element, analysis elements described in “Fuji Film Research Report, No. 40” (Fuji Photo Film Co., Ltd., issued in 1995) p. 83, “Clinical Pathology, Extra Edition, Special feature No. 106, Dry Chemistry-New Development of Simple Test” (Publication Society of Clinical Pathology, issued in 1997), etc., can be named.

A large number of multilayer dry analysis elements have been already developed and become commercial, and FUJI DRI-CHEM (manufactured by Fuji Photo Film Co., Ltd.) or the like is an example. In the invention, such a multilayer dry analysis element is used or a part thereof can also be used.

The multilayer dry analysis element may be in a mode wherein the multilayer dry analysis element is connected to the flow channel or in a mode wherein the multilayer dry analysis element is incorporated in the flow channel if the multilayer dry analysis element is in contact with at least one of the flow channels. To use a plurality of multilayer dry analysis elements, they may be collected in one place connected in the flow channel or may be arranged separately.

Often, the multilayer dry analysis element has a spreading layer for spreading blood or blood constituents in the horizontal direction on the top layer. In the invention, however, such a spreading layer is not necessarily required.

Next, the microchemical device and (a) one or more flow channels through which a specimen passes, which is another element of the microchemical device, has will be discussed.

<Microchemical Device and (a) One or More Flow Channels Through which a Specimen Passes>

The microchemical device in the invention means a device having a flow channel (also called a channel) with equivalent diameter of 1 mm or less.

The equivalent diameter mentioned in the invention is a term generally used in the field of mechanical engineering. When a circular pipe equivalent to piping of an arbitrary cross-sectional shape (corresponding to flow channel in the invention) is assumed, the diameter of the equivalent circular tube is called the equivalent diameter. Using A: Cross-sectional area of piping and p: Wetted perimeter length (peripheral length) of piping, deg: Equivalent diameter is defined as deg=4A/p. To apply to a circular pipe, the equivalent diameter matches the circular pipe diameter. The equivalent diameter is used to estimate the flow or heat transfer characteristic of the piping based on the data of the equivalent circular pipe and represents the spatial scale of phenomenon (representative length). The equivalent diameter becomes deg=4a²/4a=a in a square pipe with one side a and deg=2h in a flow between parallel planes with path length h. This topic is described in detail in “Kikaikougaku jiten” ((sha) Nihon kikaigakkaihen 1997, Maruzen (kabu)).

The equivalent diameter of the flow channel used in the invention is 1 mm or less; preferably it is 10-500 μm and particularly preferably 20-300 μm.

The length of the flow channel is not limited; preferably it is 1 mm-10000 mm and particularly preferably 5 mm-100 mm.

Preferably, the width of the flow channel used in the invention is 1-3000 μm, more preferably 10-2000 μm, and furthermore preferably 50-1000 μm. If the width of the flow channel is in the range mentioned above, a specimen of blood, etc., less receives resistance from the wall of the flow channel and flowability is less degraded and the specimen amount can be minimized.

The number of flow channels may be one or the flow channel may take two or more branches matching the number of elements placed in the microchemical device. The flow channel can adopt any shape such as a linear shape or a curved shape; preferably it is a linear shape.

The microchemical device (hereinafter, also called microreactor) of the invention and the flow channel can be prepared on a solid substrate according to a microfabrication technique.

Metal, silicon, Teflon, glass, ceramics, plastic, etc., can be named as an example of material used as the solid substrate. Among them, metal, silicon, Teflon, glass, and ceramics are preferred from the viewpoints of heat resistance, pressure resistance, solvent resistance, and light transmission; glass is particularly preferred.

As the microfabrication technique for preparing the flow channel, the methods described in “Microreactor—shin'jidaino gouseigijyutu—(2003, published by CMC, supervised by YOSHIDA Jun'ichi, Kougakuken'kyuuka professor in Kyoto University graduate school), “Bisaikakougijyutu ouyouhen—Photonics, electronics, mechatronics henoouyou—(2003, published by NTS, edited by koubun'shigakkai gyoujiiin'kai), etc., for example, can be named.

Representative methods are as follows: LIGA technique using X-ray lithography, high-aspect-ratio photolithography using EPON SU-8, microelectric discharge machining (μ-EDM), high-aspect-ratio machining of silicon by Deep RIE, Hot Emboss machining, stereo lithography, laser machining, ion beam machining, mechanical microcutting using a microtool made of a hard material such as diamond, and the like. These techniques may be used alone or in combination. The preferred microfabrication techniques are LIGA technique using X-ray lithography, high-aspect-ratio photolithography using EPSON su-8, microelectric discharge machining (μ-EDM), and mechanical microcutting.

The flow channel used in the invention can also be prepared by using a pattern formed using a photoresist on a silicon wafer as a mold and pouring resin thereinto and hardening it (molding method). For the molding method, silicon resin represented by dimethylpolysiloxane (PDMS) or its derivatives can be used.

To assemble the microchemical device of the invention, a joining technique can be used. Usual joining techniques are roughly classified into solid-phase joining and liquid-phase joining. Representative joining methods generally used are pressure welding and diffusion welding (joining) as solid-phase joining and welding, eutectic bonding, soldering, adherence, etc., as liquid-phase joining.

Further, to assemble the microchemical device, a highly accurate joining method keeping the dimension accuracy without involving destruction of the microstructure of the flow channel, etc., caused by deterioration or large deformation of material by high-temperature heating is desired; as the technique, silicon direct joining, anodic joining, surface activation joining, direct joining using hydrogen bond, joining using HF water solution, Au—Si eutectic bonding, void-free adherence, etc., can be named.

A specimen moves from the flow channel to the multilayer dry analysis element. As a method for handling a specimen in the flow channel, namely, fluid, preferably a continuous flow method, a liquid drop (liquid plug) method, a drive method, or a drive method using capillarity is used.

In fluid control of the continuous flow method, the inside of the flow channel of the microreactor needs to be fully filled with fluid and it is a common practice to drive the whole fluid by a pressure source of a syringe pump, etc., externally provided. The continuous flow method can provide a control system by comparatively easily setting.

In the liquid drop (liquid plug) method, drops partitioned by air are moved in the reactor or in the flow channel to the reactor, and each drop is driven by air pressure. In the liquid drop method, the reactor system needs to be provided internally with a vent structure for letting air between drops and a flow channel wall or between drops escape to the outside as required, a valve structure for keeping the pressure in the branch flow channel independently of other portions, and the like. To control the pressure difference for manipulating drops, it is necessary to construct an external pressure control system made up of a pressure source, a switching valve, etc. In the liquid drop method, multi-step operation can be performed in such a manner that drops are manipulated separately and several reactions are executed in order, and the flexibility of the system configuration increases.

As the drive method, an electric drive method of applying a high voltage across the flow channel (passage) for generating an electroosmotic flow, thereby moving fluid, a pressure drive method of providing an external pressure source and applying pressure to fluid for moving the fluid, and a drive method using capillarity are generally widely used.

In the electric drive method, it is known that as behavior of fluid, the flow velocity profile becomes a flat distribution in the cross section of the flow channel. In the pressure drive method, it is known that as behavior of fluid, the flow velocity profile becomes like a hyperbola, namely, a distribution wherein the velocity in the center of the flow channel is high and that in the wall face portion is low in the cross section of the flow channel. Thus, the electric drive method is preferred to the pressure drive method for the purpose of moving fluid with the shape of a sample plug, etc, kept.

The electric drive method requires that the inside of the flow channel be filled with fluid, namely, takes the mode of the continuous flow method. Since fluid can be manipulated by electric control, comparatively complicated processing can be performed in such a manner that the mixing ratio of two types of solutions is continuously changed, thereby producing a concentration gradient with time, for example.

In the pressure drive method, control can be performed without being affected by the electric nature proper to fluid. The application range of the pressure drive method is wide because the secondary effect of heat generation, electrolysis, etc., need not be considered and the effect on the substrate scarcely exists. The pressure drive method requires that an external pressure source be provided.

In the invention, an appropriate specimen moving method can be selected matching the type of specimen and the inspection items. Among the methods, the liquid drop (liquid plug) method or the drive method using capillarity is preferred. More preferably, the air pressure in the liquid drop (liquid plug) method is made negative pressure; particularly preferably negative pressure produced by suction of air.

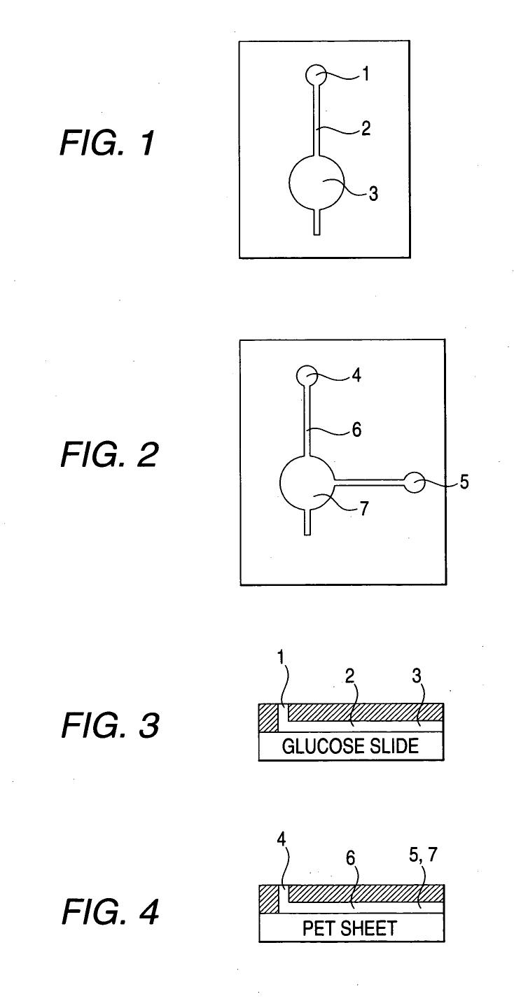

In the invention, one or more flow channels through which a specimen passes are in contact with the multilayer dry analysis element as described above. The simplest embodiment is shown in FIG. 1.

A specimen pouring port 1 may be any if it enables a specimen to be poured. The poured specimen passes through a flow channel 2 and is introduced into a reaction port 3. The reaction port 3 is in contact with the multilayer dry analysis element and reacts with a reagent.

The invention does not fall within the range of FIG. 1, needless to say.

<Specimen>

As a specimen that can be analyzed using the microchemical device of the invention, liquid constituents obtained from a human being, such as blood or urine can be named. Application of the microchemical device of the invention is preferred because the microchemical device can rapidly measure trace constituents of body fluid from the liquid constituents. Protein, enzyme, DNA, hormone, receptor, ligand, and other trace constituents existing in body fluid can be measured from blood, urine, etc. If the specimen is whole blood, preferably the whole blood as the specimen is subjected to pretreatment by a pretreatment element, leading to more rapid measurement. The pretreatment element is an element used in a blood cell separating step, for example, and as the step, any separation step usually used in the field, such as filtration or centrifugal separation. Particularly, filtration is preferred; preferably a filter material is used as the pretreatment element. It is desirable that the pretreatment element should be built in the microchemical device of the invention in one piece.

The specimens that can be analyzed using the microchemical device of the invention are not only body fluid, but also a wide range of specimens in food analysis, environment analysis, cell analysis, etc., needless to say.

### EXAMPLES

<PDMS Concave Form Preparation>

Spin coating of SU-8 of a thick film photoresist was executed on a silicon wafer to produce film thickness 100 μm. After preheating for one hour at 90° C., UV light was applied through a mask on which flow channel pattern (1) corresponding to FIG. 1 is drawn, and the light application portion was hardened for one hour at 90° C. Dissolution removal of unhardened portion was executed with propylene glycol monomethyl ether acetate (PGMEA) and the resultant portion was washed in water and then was dried for use as silicon wafer/SUS convex form.

PDMS (Du pont Sylgard/curing liquid=10/1 mixing liquid) is poured onto the silicon wafer convex form and was hardened for two hours at 80° C. and then was softly peeled off from the silicon wafer convex form to prepare PDMS concave form shown in FIG. 1.

Next, through a mask on which flow channel pattern (2) corresponding to FIG. 2, silicon wafer/SU8 convex form was prepared and then PDMS concave form shown in FIG. 2 was prepared as with flow channel pattern (1).

Adjustment was made so that specimen pouring port (1, 4) and reagent pouring port (5) become 1 mm in diameter, reaction port (3, 7) becomes 2 mm in diameter, and flow channel (2, 6) becomes 200 μm in width and 80 μm in depth.

### Example

Measurement on multilayer dry analysis element Spreading layer in glucose slide of FUJI DRI-CHEM (manufactured by Fuji Photo Film Co., Ltd.) was stripped off and the PDMS concave form in FIG. 1 was put thereon to form multilayer dry analysis element chip (1).

Glucose of 100 mg/dl was poured from the specimen pouring port 1 and thermal insulation was conducted for five minutes on a dry bath at 37° C. and then color development of the reaction port 3 was measured under a digital stereoscopic microscope (Keyence Digital Microscope).

### Comparative Example

### Measurement on Comparative Example Chip

PDMS concave form in FIG. 2 was put on a polyethylene terephthalate sheet (PET sheet) 180 μm in thickness to form comparative example chip (2).

A glucose solution of 100 mg/dl was poured from the specimen pouring port 4 and a glucose coloring reagent (GLUCOSE CII-TEST WAKO, manufactured by Wako Pure Chemicals) was poured from the specimen pouring port 5 and thermal insulation was conducted for five minutes on a dry bath at 37° C. and then color development of the reaction port 7 was measured under the digital stereoscopic microscope (Keyence Digital Microscope).

Result:

In the multilayer dry analysis element chip of example 1, uniform color development was observed on the full face of the reaction port 3, but in comparative example 2, color development unevenness was observed in the reaction port 7. The reflection density was calculated from the obtained data.

The result is listed in Table 1

TABLE 1

| Slide | Reflection density (A. U.) |
|---|---|
| Example (reaction port 3) | 1.8 |
| Comparative example (reaction port 7) | 0.7 |

As described above, it is obvious that detection by the chip having the microflow channel and the multilayer dry analysis element of the invention is effective and based on this, it is also obvious that detection by the multilayer dry analysis element in the microchemical device is effective.

This application is based on Japanese patent application JP 2004-066800, filed on Mar. 10, 2004, the entire content of which is hereby incorporated by reference, the same as if set forth at length.

1. An integrated microchemical device for analyzing one or more constituents in a specimen, said integrated microchemical device comprising:
(a) one or more flow channels through which a specimen passes; and
(b) a multilayer dry analysis element that can come in contact with at least one of the one or more flow channels and analyze the one or more constituents of the specimen.

2. The integrated microchemical device as claimed in claim 1, wherein the one or more flow channel has a width of 1 to 3000 μM.

3. The integrated microchemical device as claimed in claim 1, which further comprises a pretreatment element for pretreating a specimen.

4. An method of analyzing one or more constituents in a specimen, which comprises using the integrated microchemical device described in claim 1.
